(19) United States
(12) Patent Application Publication
(10) Pub. No.: US 2012/0115806 A1
(43) Pub. Date: May 10, 2012

(54) Antitumor Combination Including Cabazitaxel and Capecitabine

(75) Inventor: Emmanuelle MAGHERINI, Ris Orangis (FR)

(73) Assignee: SANOFI, Paris (FR)

(21) Appl. No.: 13/289,250

(22) Filed: Nov. 4, 2011

Related U.S. Application Data
(63) Continuation of application No. PCT/FR2010/050873, filed on May 6, 2010.

(30) Foreign Application Priority Data
May 6, 2009 (FR) 0902189
May 11, 2009 (FR) 0902264

(51) Int. Cl. A61K 31/7068; A61P 35/00

(52) U.S. Cl. 514/49

(57) ABSTRACT

The invention relates to a method of treatment of metastatic breast cancer in a patient progressing after a previous anthracycline and/or taxane treatment, said method comprising administering to said patient a combination of antitumor agents comprising cabazitaxel and capecitabine, wherein each of said antitumor agents, independently, may be in the form of a base, in the form of a pharmaceutically acceptable acid salt or in the form of a hydrate or solvate.

The present invention relates to an antitumor combination combining cabazitaxel and capecitabine in the treatment of metastatic breast cancer in patients progressing after prior treatment with anthracyclines and taxanes.

### PRIOR ART AND TECHNICAL PROBLEM

Breast cancer affects a large part of the female population throughout the world: 1.15 million cases worldwide in 2002; it is predicted that it will affect 1.4 million cases in 2010 (CA cancer J. Clin. 2005, 55, 74-108). It is the most common cancer in women.

Metastatic breast cancer (MBC) is generally treated with anthracycline- and taxane-based chemotherapy (“Concise Review for clinicians: advances in screening, diagnosis and treatment of breast cancer” Mayo clinic proceedings 2004, 76, 810-816).

The cancer can become resistant to the agents used, in particular to taxanes, which limits the possible treatment options. Several mechanisms of taxane resistance have been described (expression of P-glycoprotein P-gp, mdr-1 gene, modified taxane Metabolism, tubulin gene mutation, etc.): see Drug Resistance Updates 2001, 4(1), 3-8; J. Clin. Onc. 1999, 17(3), 1061-1070.

For patients in whom the cancer has progressed after a previous treatment based on anthracyclines and/or taxanes (75% of patients develop resistance to this treatment), capecitabine in monotherapy or the combination combining capecitabine and docetaxel is indicated (J. Clin. Onc. 2002, 20(12), 2812-2823).

It has also been observed that cabazitaxel (or XRP6258) can be effective in the treatment of taxane-resistant metastatic breast cancer (“A multicenter phase II study of XRP6258 administered as a 1-h i.v. infusion every 3 weeks in taxane-resistant metastatic breast cancer patients” Ann. Oncol. 2008, 19(9), 1547-1552).

Furthermore, in the conclusion of the abstract entitled “In vitro induction of Thymidine Phosphorylase by XRP6258, a new taxoid” presented at the French Pharmacology Society Conference in Clermont-Ferrand from Apr. 9 to 11, 2008, the following is specified: “XRP6258 induces TP expression, especially with MCF-7 breast carcinoma cells. This induction might be clinically relevant in the field of XRP6258/capecitabine combination, assessed in patients with breast cancer, as predictive of an increased cytotoxicity in the tumor cells for the combination”.

There is still a need to find and optimize novel therapeutic options in patients in whom the cancer has progressed after previous treatment with anthracyclines and taxanes.

The present invention meets this need by providing a novel antitumor pharmaceutical combination comprising cabazitaxel and capecitabine, for which it has been necessary to determine the doses of each drug and the suitable administration scheme, so as to obtain a well-tolerated combination which does not exacerbate the toxicity of each of the two antitumor agents and which allows the treatment of patients progressing after a prior treatment with anthracyclines and taxanes, in order to evaluate the antitumor activity thereof.

### BRIEF DESCRIPTION OF THE INVENTION

The invention relates to an antitumor pharmaceutical combination comprising cabazitaxel having the formula

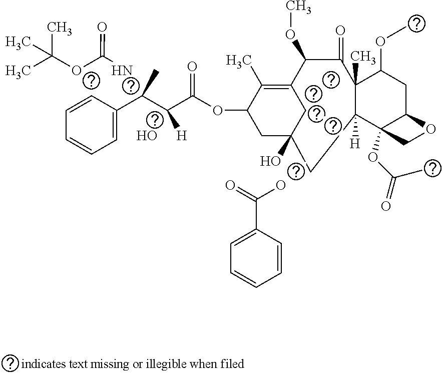

and capecitabine having the formula

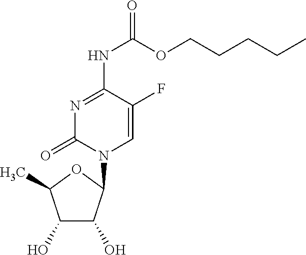

wherein both of said antitumor agents may be in the form of a base, in the form of a pharmaceutically acceptable acid salt or in the form of a hydrate or of a solvate, intended for the treatment of metastatic breast cancer in patients progressing after a prior treatment with anthracyclines and taxanes.

Cabazitaxel can in particular be in the form of an acetone solvate. More particularly, the acetone solvate of cabazitaxel contains between 5 and 8% by weight of acetone, preferably between 5 and 7%.

The combination comprises an effective amount of cabazitaxel and an effective amount of capecitabine.

The cabazitaxel can be administered at a dose (defined for each administration) of between 15 and 25 mg/m².

The capecitabine can be administered twice a day at a dose (defined for each administration) of between 675 and 1250 mg/m², more especially between 825 and 1000 mg/m².

The cabazitaxel can be administered by infusion at a dose of between 15 and 25 mg/m² and the capecitabine is administered orally twice a day for 14 days at a dose (defined for each administration) of between 675 and 1250 mg/m², more especially between 825 and 1000 mg/m², this cycle of administration of the two antitumor agents being repeated with a gap between two administrations of cabazitaxel of 3 weeks, which gap can be extended by 1 to 2 weeks depending on the tolerance to the preceding administration of cabazitaxel.

The invention also relates to the use of cabazitaxel and capecitabine of formula for preparing the abovementioned antitumor pharmaceutical combination.

### DESCRIPTION OF THE INVENTION

Definitions
pharmaceutically acceptable acid: organic or inorganic acid having a low toxicity (see Pharmaceutical salts J. Pharm. Sci. 1977, 66, 1-19);
effective amount: amount of a pharmaceutical compound producing an effect on the cancer treated.

As regards cabazitaxel, it belongs to the taxoid family and has the formula:

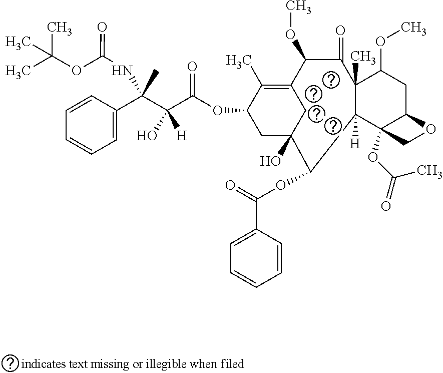

It has the chemical name: 4α-acetoxy-2α-benzoyloxy-5β,20-epoxy-1β-hydroxy-β,10β-dimethoxy-9-oxotax-11-en-13α-yl (2R,3S)-3-tert-butoxycarbonylamino-2-hydroxy-3-phenylpropionate. This compound and a method of preparation are described in document WO 96/30355. Cabazitaxel can be administered in the form of a base (cf. formula above), in the form of a pharmaceutically acceptable acid salt or in the form of a hydrate. It can also be a solvate, i.e. a molecular complex characterized by the incorporation of the crystallization solvent into the crystal of the molecule of the active ingredient (in this respect, see page 1276 of J. Pharm. Sci. 1975, 64(8), 1269-1288). In particular, it may be an acetone solvate, more particularly the one described in WO 2005/028462. It may be an acetone solvate of cabazitaxel containing between 5 and 8% by weight of acetone, preferably between 5 and 7% (% signifies acetone content/acetone+cabazitaxel content×100). An average value of the acetone content is 7%, which represents more or less acetone stoichiometry, which is 6.5% for a solvate comprising one acetone molecule. The procedure described below makes it possible to prepare an acetone solvate of cabazitaxel:

940 ml of purified water are added, at 20±5° C. ambient temperature, to a solution of 207 g of 4-acetoxy-2α-benzoyloxy-5β,20-epoxy-1-hydroxy-7β,10β-dimethoxy-9-oxotax-11-en-13α-yl, at approximately 92% by weight in approximately 2 liters of acetone, and then seeding is carried out with a suspension of 2 g of 4-acetoxy-2α-benzoyloxy-5β,20-epoxy-1-hydroxy-7β,10β-dimethoxy-9-oxotax-11-en-13α-yl (2R,3S)-3-tert-butoxycarbonylamino-2-hydroxy-3-phenylpropionate isolated in acetone/water in a mixture of 20 ml of water and 20 ml of acetone. The mixture is left to stir for approximately 10 to 22 hours and 1.5 liters of purified water are added over the course of 4 to 5 hours. The mixture is left to stir for 60 to 90 minutes and then the suspension is filtered under reduced pressure. The cake is washed on a filter with a solution prepared from 450 ml of acetone and 550 ml of purified water and then oven-dried at 55° C. under reduced pressure (0.7 kPa) for 4 hours. 197 g of 4-acetoxy-2α-benzoyloxy-5β,20-epoxy-1-hydroxy-7β,10β-dimethoxy-9-oxotax-11-en-13α-yl (2R,3S)-3-tert-butoxycarbonylamino-2-hydroxy-3-phenylpropionate acetone containing 0.1% of water and 7.2% of acetone are obtained (theoretically 6.5% for a stoichiometric solvate).

Cabazitaxel is administered parenterally, such as by intravenous administration, as a bolus or by infusion. A galenical form of cabazitaxel suitable for administration by infusion is the form wherein the cabazitaxel is in solution in water in the presence of excipients chosen from surfactants, cosolvents, glucose or sodium chloride, etc. For example, a galenical form of cabazitaxel can be prepared by dilution of a premix solution of cabazitaxel contained in a sterile vial (80 mg of cabazitaxel+2 ml of solvent+polysorbate 80) with a sterile vial containing a solution of 6 ml of water and ethanol (13% by weight of 95% ethanol) so as to obtain 8 ml of a solution ready for redilution in a drip bag. The concentration of cabazitaxel in this ready-for-redilution solution is approximately 10 mg/ml. The infusion is then prepared by injecting the appropriate amount of this ready-for-redilution solution into the drip bag containing water and glucose (approximately 5%) or sodium chloride (approximately 0.9%).

As regards capecitabine (CAS RN 154361-50-9), it is sold by the company Roche under the trademark Xeloda®. It is a prodrug of 5-fluorouracil:

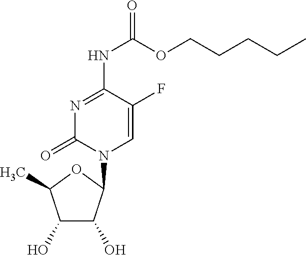

and has the chemical name: 5′-deoxy-5-fluoro-N4-(pentyloxycarbonyl)cytidine N-[1-(5-deoxy-beta-D-ribofuranosyl)-5-fluoro-2-oxo-1,2-dihydropyrimidin-4-yl]carbamic acid pentyl ester. This compound is described in EP 0602454 or U.S. Pat. No. 5,472,949. Capecitabine can be administered in the form of a base (cf. formula above), in the form of a pharmaceutically acceptable acid salt or in the form of a hydrate or of a solvate.

A galenical form of capecitabine suitable for oral administration is, for example, the product sold under the trademark Xeloda® in the form of tablets containing 150 or 500 mg of capecitabine and anhydrous lactose as excipient.

As regards the combination, it consists in combining cabazitaxel and capecitabine in the form of two distinct pharmaceutical preparations. The combination can be used in the treatment of metastatic breast cancer, in particular for patients who still have the cancer after a treatment based on anthracyclines and/or taxanes.

The combination is administered repeatedly according to a protocol which depends on the patient to be treated (body surface area, tolerance to the previous cycle, etc.). The cabazitaxel can be administered to the patient by infusion according to an intermittent scheme with a gap between each administration of 3 weeks, that can be extended by 1 to 2 weeks depending on the tolerance to the preceding administration. The capecitabine can, for its part, be administered daily, for example in the form of two intakes per day, for a period of 14 days. During a cycle, the 1st intake of capecitabine can coincide with the administration of cabazitaxel.

An example of the protocol is as follows: the cabazitaxel is administered by infusion over a period of approximately 1 hour on a given day D1 (1st day of the cycle). The capecitabine is administered orally twice a day, in the morning and evening, from day D1 to day D14 (from the 1st to the 14th day of the cycle). This cycle consisting in administering both the cabazitaxel (on D1) and the capecitabine (from D1 to D14) is then repeated with a gap of 3 weeks (extendable by 1 to 2 weeks).

The cabazitaxel and capecitabine doses administered each time to the patient depend on various parameters: body surface area, tolerance to the previous cycle, etc. The cabazitaxel can be administered at a dose (defined for each administration) of between 15 and 25 mg/m². The capecitabine can be administered twice a day at a dose (defined for each administration) of between 675 and 1250 mg/m², more especially between 825 and 1000 mg/m².

Preferably, the recommended dose is 20 mg/m² of cabazitaxel on the first day of treatment and 2×1000 mg/m²/day of capecitabine from the first to the fourteenth day, this cycle of administration of the two antitumor agents being repeated with a gap between two cabazitaxel administrations of 3 weeks, which gap can be extended by 1 to 2 weeks depending on the tolerance to the previous administration of cabazitaxel.

### EXAMPLE

A phase I/II study was carried out in four study centers in Europe.

Part 1: the maximal administered dose (MAD) and the recommended dose (RD) of cabazitaxel in combination with capecitabine were determined.

Part 2: the antitumor activity and the characterization of the tolerance profile were determined at the recommended dose.

The pharmacokinetics (PK), including the drug interaction study, were also studied.

Patients

Main Inclusion Criteria for Patients

The main inclusion criteria were an age of 18 or older, a histologically proven metastatic or locally recurrent breast cancer that was inoperable, an Eastern Cooperative Oncology Group performance status (ECOG PS) of 0 to 2, prior exposure to taxanes and to anthracyclines, and adequate hematological, renal and hepatic function. For part 2, the patients selected had to have at least 1 measurable lesion according to the RECIST guidelines (J Natl Cancer Inst 2000, 92, 205-216).

The main exclusion criteria were a simultaneous cancer, more than one chemotherapy treatment for metastatic cancer, prior exposure to capecitabine, or uncontrolled significant comorbid conditions.

Thirty-three patients with metastatic breast cancer were included in the study, all previously treated with anthracyclines and taxanes: 15 patients for part 1 and 18 for part 2.

The characteristics relating to the patients treated are specified in Table I.

TABLE I

| Number of patients | 33 | |
|---|---|---|
| Median age [min-max] (must be >=18) | 55 | [34-74] |
| Diagnosis: | | |
| infiltrating ductal carcinoma | 25 | (76%) |
| infiltrating lobular carcinoma | 4 | (12%) |
| infiltrating mixed carcinoma | 4 | (12%) |
| Median time since initial diagnosis in | 4.97 | [1.2-21.5] |
| years [min-max] | | |
| Her2 Neu status by FISH | 33 | (100%) |
| negative | | |
| Hormone receptor | | |
| ER+ and/or PgR+ | 29 | (88%) |
| ER− and PgR− | 4 | (12%) |
| ECOG PS at entry into the study | | |
| 0 | 20 | (61%) |
| 1 | 13 | (39%) |
| Median number of metastatic organs | 3 | (1-6) |
| Disease status at beginning of treatment | metastatic: | |
| main organs invaded: | 33 | (100%) |
| bone | 27 | (82%) |
| liver | 20 | (61%) |
| distant lymph nodes | 13 | (39%) |
| lung | 10 | (30%) |
| pleura | 7 | (21%) |
| regional lymph nodes | 4 | (12%) |
| others^{a} | 6 | (18%) |
| Prior treatment | | |
| Number of lines of prior chemotherapy for | | |
| advanced disease: | | |
| 0 | 5 | (15%) |
| 1 | 28 | (85%) |

^{a}includes adrenal, soft tissue, peritoneum, pericardial effusion.
ECOG PS: Eastern Cooperative Oncology Group performance status;
ER: estrogen receptor;
PgR: progesterone receptor;
HER2: human epidermal growth factor receptor.

Pharmacokinetics:

The pharmacokinetic parameters were calculated for cabazitaxel, capecitabine and their metabolites (Cₘₐₓ, Tₘₐₓ, AUC₀₋ₗₐₛₜ, AUC, t_{1/2λ}).

Blood samples were collected at various times in part 1 of the study and tested by liquid chromatography coupled to mass spectrometry methods.

The pharmacokinetic analysis did not reveal any apparent interaction between cabazitaxel and capecitabine; the pharmacokinetics of cabazitaxel and of its metabolite do not appear to be affected by the coadministration of capecitabine, and vice versa.

Part 1: determination of the maximal administered dose (MAD) and of the recommended dose (RD) of cabazitaxel in combination with capecitabine.

The dose-limiting toxicities (DLTs), i.e. the list of events to be monitored, which make it possible to guide the dose escalation, were first of all predefined in the protocol in accordance with the NCI-CTCAE classification scale, version 3.

The increase in doses was studied in groups of three patients as long as no DLT was observed. If a DLT was noted in a patient, the group was extended to six patients, the MAD being reached if at least 2 patients suffered a DLT. The RD was defined as the highest dose at which less than 33% of the patients exhibited a DLT.

First Dose Level:
3 patients—cabazitaxel 20 mg/m² (D1) and capecitabine 825 mg/m² twice a day (D1-14)
1 DLT of grade 4 neutropenia type for more than 7 days in one patient was observed
group extended to 6 patients without further DLTs.

Second Dose Level:
3 patients—cabazitaxel 20 mg/m² (D1) and capecitabine 1000 mg/m² twice a day (D1-14)
no DLT observed.

Third Dose Level:
3 patients—cabazitaxel 25 mg/m² (D1) and capecitabine 1000 mg/m² twice a day (D1-14)
1 DLT of grade 4 neutropenia type for more than 7 days in one patient was observed
group extended to 6 patients—a second DLT of the same type in another patient was observed.

The MAD was consequently defined as being: cabazitaxel 25 mg/m² and capecitabine 1000 mg/m².

The RD was consequently defined as being: cabazitaxel 20 mg/m² and capecitabine 1000 mg/m².

Part 2: Study of the antitumor activity and characterization of the tolerance profile at the recommended dose

The patients included in part 2 of the study were treated with the RD.

The main criterion for evaluating efficacy was the objective response rate (ORR).

The objective response rate is defined, according to the RECIST guidelines (J Natl Cancer Inst 2000, 92, 205-216), as the proportion of patients with a confirmed complete response (CR) or partial response (PR), divided by the total number of patients in the analysis population.

The secondary criteria for evaluating efficacy were the duration of response (DR) and the time to progression (TTP).

The time to progression (TTP) is defined, according to the RECIST guidelines (J Natl Cancer Inst 2000, 92, 205-216), as the time elapsed between the first date of administration of the combination and the date of the first documentation of progression of the disease.

The duration of response (DR) is defined, according to the RECIST guidelines (J Natl Cancer Inst. 2000, 92, 205-216), as the time elapsed between the date of the first documentation of an objective response (CR or PR) and the date of the first documentation of progression of the disease or the occurrence of a death.

The capecitabine is administered orally twice a day, in the morning and evening, from day D1 to day D14 (from the 1st to the 14th day of the cycle). Two hours after the administration in the morning, the cabazitaxel is administered by infusion (iv) over a period of approximately 1 hour on day D1 (1st day of the cycle). This cycle consisting in administering both the cabazitaxel (on D1) and the capecitabine (from D1 to D14) is then repeated every three weeks.

Table II gives details of a concrete example of a cycle.

TABLE II

| Time | Day D1 | Days D2 to D14 |
|---|---|---|
| 7:30 | Breakfast | Breakfast |
| 8:00 | Capecitabine (oral) | Capecitabine (oral) |
| 10:00 | Cabazitaxel (infusion) | |
| 19:30 | Evening meal | Evening meal |
| 20:00 | Capecitabine (oral) | Capecitabine (oral) |

The doses could be reduced and the treatment cycle duration could be extended in the case of a severe adverse event (AE). The treatment was continued until disease progression, the presence of an unacceptable AE or the withdrawal of patient consent.

A physical examination, complete differential leukocyte and blood counts, and blood chemistry analyses were carried out before the recruitment of the patients and regularly during treatment. Tumor evaluation by radiology was carried out at recruitment of the patients and every 6 weeks. The responses were confirmed by 2 evaluations at least 4 weeks apart. Supplementary data were collected every 6 weeks until the date the study was stopped.

Table Ill summarizes the treatment characteristics of the patients at the various dose levels tested.

TABLE III

| | | DL1 | DL2 | DL3 | |
|---|---|---|---|---|---|
| | | (20 mg/m² | (20 mg/m² | (25 mg/m² | |
| | | cabazitaxel + 2 × | cabazitaxel + 2 × | cabazitaxel + 2 × | |
| | | 825 mg/m²/day | 1000 mg/m²/day | 1000 mg/m²/day | |
| | | capecitabine for | capecitabine for | capecitabine for | |
| Dose | | 14 days) | 14 days) | 14 days) | Total |
| Number of patients | | 6 | 21^{a} | 6 | 33 |
| Number of cycles | | 30 | 112 | 36 | 178 |
| Cabazitaxel | total | 30 | 109 | 36 | 175 |
| | median | 4.5 [2-12] | 5 [2-13] | 5.5 [4-9] | 5 [2-13] |
| | [min-max] | | | | |
| Capecitabine | total | 30 | 112 | 36 | 178 |
| | median | 4.5 [2-12] | 6 [2-13] | 5.5 [4-9] | 5 [2-13] |
| | [min-max] | | | | |
| Number of | | 1 | 10 | 5 | 16 |
| patients with | | | | | |
| a delayed | | | | | |
| treatment | | | | | |
| Number of | Cabazitaxel | 1 | 4 | 5 | 10 |
| patients with | Capecitabine | 0 | 3 | 4 | 7 |
| dose | | | | | |
| reduction | | | | | |
| Median | Cabazitaxel | 0.97 [0.86-1.01] | 0.97 [0.69-1.00] | 0.82 [0.74-0.91] | 0.96 [0.69-1.01] |
| relative dose | Capecitabine | 0.92 [0.64-1.00] | 0.89 [0.55-1.02] | 0.83 [0.71-1.03] | 0.87 [0.55-1.03] |
| intensity | | | | | |

^{a}including 3 patients from part 1 and 18 additional patients

Tolerance

The most common adverse events (AE) were gastrointestinal problems, fatigue, hand-foot syndrome and hematological toxicity, which corresponds to the profile generally expected for a taxane-capecitabine combination.

Table IV gives the results of antitumor activity at the various dose levels tested.

TABLE IV

| | DL1 | DL2 | DL3 | |
|---|---|---|---|---|
| | (20 mg/m² | (20 mg/m² | (25 mg/m² | |
| | cabazitaxel + 2 × | cabazitaxel + 2 × | cabazitaxel + 2 × | |
| | 825 mg/m²/day | 1000 mg/m²/day | 1000 mg/m²/day | |
| Results | capecitabine for | capecitabine for | capecitabine for | |
| number of patients (%) | 14 d) | 14 d) | 14 d) | Total |
| Number of patients | 6 | 21^{a} | 6 | 33 |
| Complete response | 0 | 1 (5)^{c} | 1 (17) | 2 (6) |
| Partial response | 0 | 4 (19) | 1 (17) | 5 (15) |
| Stabilization | 5 (83) | 11 (52) | 4 (67) | 20 (64)^{b} |
| Progression | 1 (17) | 5 (24) | 0 | 6 (18) |
| ORR, % (95% CI) | — | 24 (8-47) | — | — |
| Mean DR, month (interval) | — | 3.06 (2.1-8.4) | 4.42 (3.1-5.8) | 3.06 (2.1-8.4) |
| Mean TTP, month (95% CI) | | 4.9 (2.7-NA) | NA | 4.9 (3.4-NA) |

^{a}including 3 patients from part 1 and 18 additional patients
^{b}including 7 patients having an unconfirmed partial response (4 patients with cabazitaxel 20 mg/m² + capecitabine 825 mg/m²; 2 patients with cabazitaxel 20 mg/m² + capecitabine 1000 mg/m² and 1 patient with cabazitaxel 25 mg/m² + capecitabine 1000 mg/m²).
^{c}patient with a peritoneal carcinosis at entry into the study, who presented a confirmed CR.
CI: confidence interval;
NA: not available.

Part 2 of the study confirmed the feasibility of the antitumor pharmaceutical combination comprising cabazitaxel and capecitabine at the recommended dose since it was observed that the combination is well tolerated and does not exacerbate the toxicity of each of the two antitumor agents. In addition, encouraging antitumor activity signals were observed at this dose.

An antitumor activity was, moreover, observed at all the dose levels tested.

In total, 7 patients showed an objective response.

It is remarkable to note that 2 of the 4 patients who had a progression of the disease as best response with the prior taxane therapy experienced a stabilization of their cancer with the cabazitaxel and capecitabine combination, while 4 patients who had a stabilization of their disease as best response with the prior taxane therapy obtained an objective response to the treatment.

1. A method of treating metastatic breast cancer in a patient who has progressed after anthracycline and/or taxane treatment, said method comprising administering to said patient a combination of antitumor agents comprising cabazitaxel, having the formula 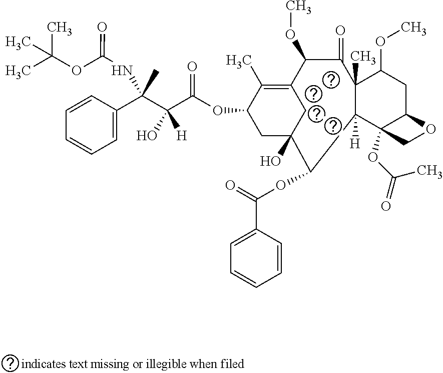
and capecitabine, having the formula 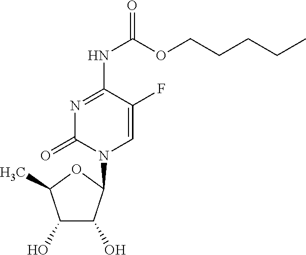
wherein each of said antitumor agents, independently, may be in the form of a base, in the form of a pharmaceutically acceptable acid salt, or in the form of a hydrate or of a solvate.

2. The method of claim 1, wherein said method comprises administration of an effective amount of cabazitaxel and an effective amount of capecitabine, wherein each of said cabazitaxel and said capecitabine, independently, may be in the form of a base, in the form of a pharmaceutically acceptable acid salt, or in the form of a hydrate or of a solvate.

3. The method of claim 2, wherein said cabazitaxel is in the form of an acetone solvate.

4. The method of claim 3, wherein said acetone solvate of cabazitaxel contains between 5% and 8% by weight of acetone.

5. The method of claim 4, wherein said acetone solvate of cabazitaxel contains between 5% and 7% by weight of acetone.

6. The method of claim 5, wherein the cabazitaxel is administered at a dose (defined for each administration) of from about 15 to about 25 mg/m².

7. The method of claim 6, wherein the capecitabine is administered twice a day at a dose (defined for each administration) of from about 675 to about 1250 mg/m².

8. The method of claim 6, in which the capecitabine is administered for 14 days.

9. The method of claim 8, wherein the cabazitaxel is administered by infusion at a dose of from about 15 to about 25 mg/m² and the capecitabine is administered orally twice a day for 14 days at a dose (defined for each administration) of from about 675 to about 1250 mg/m², this cycle of administration of the two antitumor agents being repeated with a gap between two cabazitaxel administrations of 3 weeks, which gap can be extended by 1 to 2 weeks depending on the tolerance to the preceding cabazitaxel administration.

10. The method of claim 9, wherein the capecitabine is administered orally twice a day for 14 days at a dose (defined for each administration) of from about 825 to about 1000 mg/m².

11. The method of claim 8, wherein the cabazitaxel is administered at the dose of 20 mg/m² on the first day of treatment and the capecitabine is administered at the dose of 2×1000 mg/m²/day from the first to the fourteenth day of treatment.

12. The method of claim 9, wherein, during a cycle, the first intake of capecitabine coincides with the administration of cabazitaxel.

13. The method of claim 10, wherein, during a cycle, the first intake of capecitabine coincides with the administration of cabazitaxel.

14. The method of claim 11, wherein, during a cycle, the first intake of capecitabine coincides with the administration of cabazitaxel.
